(19) United States
(12) Patent Application Publication
(10) Pub. No.: US 2017/0056083 A1
(43) Pub. Date: Mar. 2, 2017

(54) FLEXIBLE STAPLE

(71) Applicant: First Ray, LLC, Logan, UT (US)

(72) Inventors: T. Wade Fallin, Hyde Park, UT (US); Daniel J. Triplett, Providence, UT (US)

(73) Assignee: First Ray, LLC, Logan, UT (US)

(21) Appl. No.: 15/234,660

(22) Filed: Aug. 11, 2016

Related U.S. Application Data
(60) Provisional application No. 62/212,346, filed on Aug. 31, 2015.

(51) Int. Cl. A61B 17/84; A61B 17/88; A61B 17/068

(52) U.S. Cl. CPC A61B 17/846; A61B 17/068; A61B 17/8872

(57) ABSTRACT

Examples of the invention provide a staple having two or more legs joined by a flexible member and related instruments and methods.

### CROSS-REFERENCE TO RELATED APPLICATIONS

This application claims the benefit of U.S. Provisional Application No. 62/212,346, filed Aug. 31, 2015, which is hereby incorporated by reference.

### FIELD OF THE INVENTION

Examples of the invention relate generally to medical devices for connecting tissues together and more particularly to staples having two or more legs joined by a flexible member.

### BACKGROUND

Various conditions may affect skeletal joints such as the deterioration, elongation, shortening, or rupture of soft tissues, cartilage, and/or bone associated with the joint and consequent laxity, pain, and/or deformity. It is often desirable to attach a member, such as soft tissue, a graft, an implant, or other member, to an adjacent tissue, including for example a bone. It is often desirable to change the angular alignment of a bone or a portion of a bone to restore function and/or reduce pain. It likewise is often desirable to fuse a joint to fix the bones of the joint in a better angular alignment or reduce pain caused by motion at the joint. It likewise is often desirable to support a fractured bone to allow healing of the fracture to occur. To this end, various soft tissue procedures, osteotomy procedures, joint fusion procedures, fracture fixation procedures, implants and instruments have been proposed. Such procedures have been performed throughout the body to make various angular adjustments in, fuse joints associated with, and/or fuse fractures associated with tibia, fibula, femur, pelvis, humerus, ulna, radius, carpal, metacarpal, tarsal, metatarsal, phalangeal and other bones. What is needed is improved implants, instruments, and methods for performing such procedures.

### SUMMARY

Examples of the invention provide a staple having two or more legs joined by a flexible member and related instruments and methods.

In one example of the invention, a flexible bone staple includes a first member having a proximal portion and a distal portion. The distal portion includes a first leg having an elongate body extending along a first leg axis from a proximal end to a distal end and having a length measured between the proximal end and the distal end. The proximal portion includes a first extension having an elongate body extending from a proximal end to a distal end and having a length measured between the proximal end and the distal end. The distal end of the first extension is joined to the proximal end of the first leg at a first frangible junction in axial force transmitting relationship. The first member is responsive to bending to break at the frangible junction and separate the first leg and first extension. A second member is configured like the first member and the first and second member are joined by a suture having a first portion fixed to the first leg and a second portion fixed to the second leg.

In another example of the invention, a method of making a flexible bone staple includes utilizing first and second members like those of the previous example, each leg further having an axial passage extending from the proximal end of the leg at least part-way to the distal end of the leg; passing a first portion of a suture into the axial passage of the first leg and fixing the first portion of the suture to the first leg; and passing a second portion of the suture into the axial passage of the second leg and fixing the second portion of the suture to the second leg.

In another example of the invention, a driver receives the proximal end of at least one of the first and second extensions in axial force transmitting relationship.

In another example of the invention, a tamp includes first and second passages formed into the body. The second passage is formed more deeply than the first passage.

In another example of the invention, a method of using a flexible bone staple includes inserting a first leg into a first bone portion; inserting a second leg into a second bone portion, a suture extending between the first and second legs; tensioning the suture by driving at least one of the first and second legs below the surface of the first or second bone portion; and breaking first and second extensions away from the first and second legs.

In another example of the invention, a method of using a flexible bone staple includes impacting a driver body to advance first and second legs into first and second bone portions; removing the driver body; individually driving first and second extensions connected to the first and second legs to independently advance the first and second legs to independent insertion depths; moving a sleeve body proximally relative to the legs; and breaking the first and second extensions away from the first and second legs.

In another example of the invention, a method of performing an osteotomy includes cutting a bone to define first and second bone portions on either side of a cut; inserting a first leg of a bone staple into the first bone portion; inserting a second leg of the bone staple into the second bone portion, a suture extending between the first and second legs; securing the first and second bone portions together by driving at least one of the first and second legs below the surface of the first or second bone portions; and breaking first and second extensions away from the first and second legs.

### BRIEF DESCRIPTION OF THE DRAWINGS

Various examples of the invention will be discussed with reference to the appended drawings. These drawings depict only illustrative examples of the invention and are not to be considered limiting of its scope.

FIG. 1 is a perspective view of a surgical staple according to an example of the invention;

FIG. 2 is a perspective view of the surgical staple of FIG. 1;

FIG. 3 is a front view of the surgical staple of FIG. 1;

FIG. 4 is a top view of the surgical staple of FIG. 1;

FIG. 5 is a bottom view of the surgical staple of FIG. 1;

FIG. 6 is a side view of the surgical staple of FIG. 1;

FIG. 7 is a sectional view taken along line 7-7 of FIG. 6;

FIG. 8 is a detail view as indicated by reference number 8 of FIG. 7;

FIG. 9 is a perspective view of the staple of FIG. 1 after the leg extensions have been removed;

FIG. 10 is a front view of the staple of FIG. 1 after the leg extensions have been removed;

FIG. 11 is a perspective view of a driver for use with the staple of FIG. 1 according to an example of the invention;

FIG. 12 is a front view of the driver of FIG. 11;

FIG. 13 is a side view of the driver of FIG. 11;

FIG. 14 is a bottom view of the driver of FIG. 11;

FIG. 15 is a perspective view of a driving body portion of the driver of FIG. 11;

FIG. 16 is a front view of the driving body portion of FIG. 15;

FIG. 17 is a top view of the driving body portion of FIG. 15;

FIG. 18 is bottom view of the driving body portion of FIG. 15;

FIG. 19 is a side view of the driving body portion of FIG. 15;

FIG. 20 is a section view taken along line 20-20 of FIG. 19;

FIG. 21 is a perspective view of a sleeve body portion of the driver of FIG. 11;

FIG. 22 is a front view of the sleeve body portion of FIG. 21;

FIG. 23 is a top view of the sleeve body portion of FIG. 21;

FIG. 24 is bottom view of the sleeve body portion of FIG. 21;

FIG. 25 is a side view of the sleeve body portion of FIG. 21;

FIG. 26 is a section view taken along line 26-26 of FIG. 25;

FIG. 27 is a front view of an assembly of the staple of FIG. 1 with the driver of FIG. 11 according to an example of the invention;

FIG. 28 is a section view of the assembly of FIG. 27;

FIG. 29 is a perspective view of a hole forming guide for use with the staple of FIG.1 according to an example of the invention;

FIG. 30 is a top view of the hole forming guide of FIG. 29;

FIG. 31 is a side view of the hole forming guide of FIG. 29;

FIG. 32 is a front of a tamp for use with the staple of FIG. 1 according to an example of the invention;

FIG. 33 is a top view of the tamp of FIG. 32;

FIG. 34 is a bottom view of the tamp of FIG. 32;

FIG. 35 is a section view taken along line 35-35 of FIG. 32;

FIG. 36 is a perspective view of a leg setting tool for use with the staple of FIG. 1 according to an example of the invention;

FIG. 37 is a top view of the leg setting tool of FIG. 36;

FIGS. 38-44 are perspective views illustrating a surgical procedure using the staple and instruments of FIGS. 1-37 according to an example of the invention;

FIG. 45-47 are partial sectional views illustrating a surgical procedure according to an example of the invention;

FIG. 48 is a front view of a staple with more than two legs according to an example of the invention;

FIG. 49 is a front view of the hole forming guide of FIG. 29 in use with an indexing pin according to an example of the invention;

FIG. 50 is a perspective view illustrating the use of staples according to an example of the invention to attach a first member to bone;

FIG. 51 is a perspective view illustrating the use of a five-legged staple in which the suture portions connect the legs in a closed loop; and

FIG. 52 illustrates a kit according to an example of the invention.

### DESCRIPTION OF THE ILLUSTRATIVE EXAMPLES

The following illustrative examples describe methods, implants, and instruments for joining first and second tissue portions together and for joining a member to tissue. The tissue portions may be portions of the same tissue that have become separated due to a fracture or a cut. The tissue portions may be portions of different tissues. In particular, the methods, implants, and instruments are particularly well suited to joining bone portions such as portions separated due to fractures or osteotomies or in an arthrodesis performed to fuse a joint. The invention may be used on any tissue and in particular on any bone or joint including but not limited to bones such as a tibia, fibula, femur, pelvis, humerus, ulna, radius, carpal, metacarpal, tarsal, metatarsal, phalange and joints associated therewith.

The terms “suture” and “suture strand” are used herein to mean any flexible member, natural or synthetic, useful in a surgical procedure and that are easily flexed. Examples include polymer sutures, wires, surgical tapes, tissue derived strands, and other suitable flexible strands or members. Sutures may be monofilament or multi-filament structures. The term “transverse” is used herein to mean crossing as in non-parallel. The term includes, but is not limited to, perpendicular relationships.

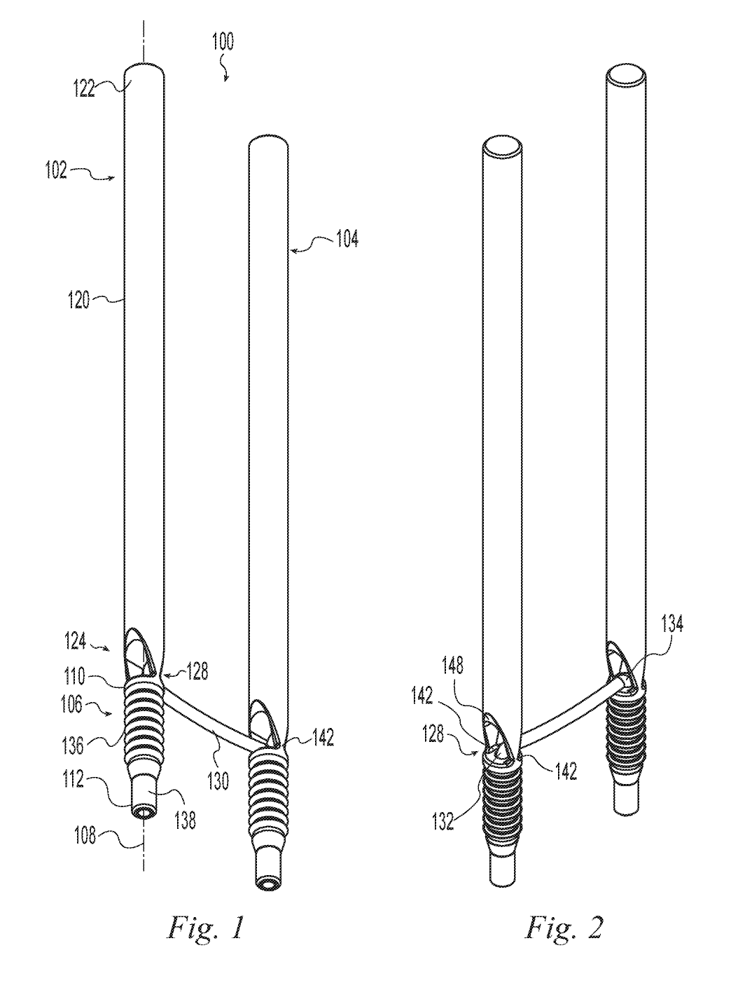

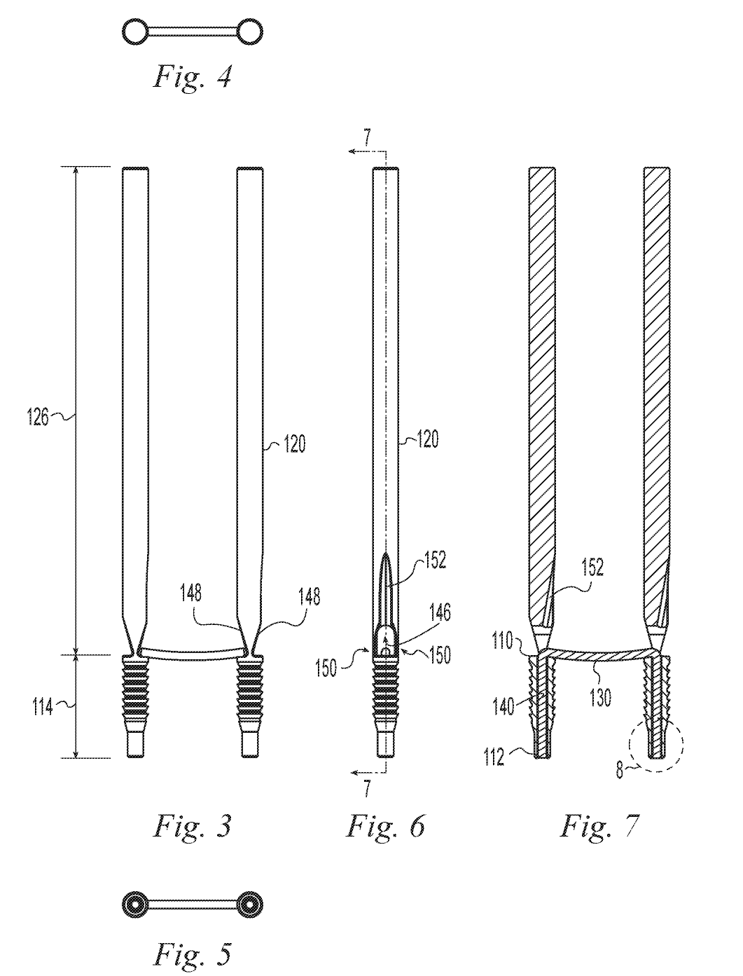

FIGS. 1-10 depict a flexible staple 100. The staple 100 includes a first member 102 and a second member 104. Each of the first and second members 102, 104 has a proximal portion and a distal portion. The distal portion includes a leg 106 having an elongate body extending along a leg axis 108 from a proximal end 110 to a distal end 112 and having a length 114 (FIG. 3) measured between the proximal and distal ends 110, 112. The proximal portion includes a first extension 120 having an elongate body extending from a proximal end 122 to a distal end 124 and having a length 126 measured between the proximal and distal ends 122, 124. The distal end 124 of the extension is joined to the proximal end 110 of the leg at a frangible junction 128 in axial force transmitting relationship along the first leg axis 108. In the illustrative example of FIGS. 1-10, the extensions 120 are coaxial with the legs 106. Each of the first and second members 102, 104 may be bent to preferentially break the member at the frangible junction 128 to separate the extension 120 from the leg 106. Preferably, the extension length 126 is equal to or greater than the leg length 114 to aid in handling the staple 100 and in breaking the frangible junction. More preferably, the extensions 120 are 1-10 times longer than the legs 106. More preferably, the extensions 120 are 2-5 times longer than the legs 106. One or more sutures 130 join the legs 106 of the first and second members 102, 104. A first portion 132 of the suture is joined to the leg of the first member 102 and a second portion 134 of the suture is joined to the leg of the second member 104. The suture 130 allows the legs to easily move relative to one another so that, for example, the suture can conform to non-flat anatomy between the legs 106 or so that the legs may be driven at varying angles or so that the legs 106 may be driven below the surface of a tissue, pulling a portion of the suture into the tissue to thereby shorten the amount of suture extending from the tissue and thus causing the legs 106 to move closer together and/or to increase tension in the suture bridging between the legs 106. Preferably, the suture 130 bridges between the legs 106 at the proximal ends 110 of the legs so that the legs 106 may be driven flush with or below the surface of the receiving tissue while a portion of the suture lies on the surface. The extensions 120 may be used to drive the legs 106 into body tissue and then the extensions 120 may be broken away from the legs 106 leaving the legs 106 embedded in the body tissue.

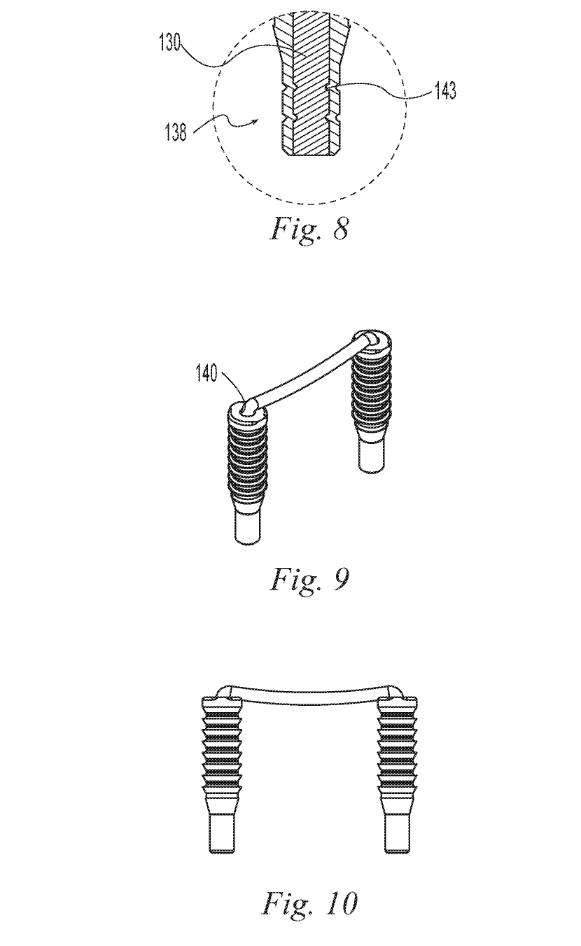

In the illustrative example of FIGS. 1-10, the legs 106 each include a barbed outer surface 136 configured for gripping bone tissue. A reduced diameter leading end portion 138 extends distally beyond the barbed outer surface 136. An axial bore 140 (FIG. 7) extends longitudinally into each leg 106 from the proximal end and receives the suture 130. Preferably, the bore 140 extends through each leg. Preferably, the suture 130 extends from the proximal end 110 of each leg 106 inside the bore 140 to the distal end 112 and bridges between the legs at the proximal ends 110. The reduced diameter leading end portion 138 provides a region for crimping the leg to secure the suture 130 inside the leg. Preferably, the leading end portion 138 has a thinner sidewall than the proximal portion of the leg and is free of barbs so that it presents a smooth outer wall for crimping. For example, as shown in FIG. 8, one or more crimps 143 may be formed in the leading end portion 138 so that the sidewall of the leg is deformed inwardly and grips the suture 130. Any suitable joining method may be used to secure the suture including crimping, adhering, knotting, molding, heat staking, pinning, and/or other joining technology. One or more joining technologies may be combined. The leading end portion 138 also aids in aligning and initiating insertion of the leg 106 into a hole formed in a tissue. Alternatively, the leg may be driven directly into a tissue without first forming a hole.

The legs 106 and extensions 120 may be integrally formed or they may be formed separately and joined at the frangible junction 128. The frangible junction may be made frangible by virtue of a reduced cross sectional area, an adhesive weaker than the joined materials, a separable joint such as a press fit joint, or by other joining technology. In the illustrative example of FIGS. 1-10, the extensions 120 are formed integrally with the legs 106 and the frangible junctions 128. For example, the legs 106 and extensions 120 may be formed by machining, molding, casting, or other forming process. In the illustrative example of FIGS. 1-10, the extensions 120 and legs 106 are machined from solid stock. The frangible junction 128 is formed by removing most of the material between the extensions 120 and legs 106 so that only narrow connections 142 remain (FIG. 2). In the illustrative example of FIGS. 1-10, a transverse arch-shaped opening 146 (FIG. 6) is formed through the extensions 120 and angled relief cuts 148 converge distally to create spaced apart tapered struts 150. The proximal portion of the leg 106 adjacent the narrow connections 142 has a cross sectional area greater than the narrow connections 142 and is stronger in bending than the narrow connections 142. Likewise, the struts 150 expand proximally such that the portion of the extension 120 adjacent the narrow connections 142 has a cross sectional area greater than the narrow connections 142 and is stronger in bending than the narrow connections 142. When the first member 102 is bent, it breaks at the narrow connections 142 to separate the extension 120 from the leg 106.

The staple 100 may be provided in multiple configurations including variation in leg number, length, width, shape (e.g. round or polygonal, with or without barbs), and material; suture number, length, width, shape (e.g. tape, round, braided, monofilament), and material; and extension length, width, shape, and material. For example, wider legs and suture may be used for larger tissues and narrower legs and suture may be used for smaller tissue. For example, for a staple to be used to attach a first bone portion to a second bone portion or to attach soft tissue to bone, the strength of the particular bone or soft tissue, the length that needs to be spanned, and the general topology of the surgical site may influence the desired staple configuration. For a given width of leg and suture it may be desirable to provide staples with varying leg length and suture length to accommodate variations between patients. For example, leg width for general orthopedic applications may range from 1 mm to 10 mm. For most situations, leg length may preferably range from 1.5 mm to 5 mm. Specifically, for example, for securing closing wedge osteotomies or securing soft tissue augmentation patches in the bones of the hands and feet, leg width preferably ranges from 1.5 mm to 3 mm. Leg lengths for general orthopedic applications may range from 3 mm to 120 mm. For most situations leg length may preferably range from 3 mm to 50 mm. Specifically, for example, for securing closing wedge osteotomies or securing soft tissue augmentation patches in the bones of the hands and feet, leg length preferably ranges from 6 mm to 12 mm. The length of suture spanning between the legs for general orthopedic applications may range from 2 mm to 100 mm. For most situations the spanning length may preferably range from 2 mm to 30 mm. Specifically, for example, for securing closing wedge osteotomies or securing soft tissue augmentation patches in the bones of the hands and feet, the spanning length preferably ranges from 4 mm to 12 mm.

The leg 106 and extension 120 may be made of metal, plastic, ceramic, tissue, and other suitable biocompatible materials. For example, metals may include stainless steel alloys, titanium, titanium alloys, cobalt-chromium steel alloys, nickel-titanium alloys, and/or others. Polymers may include nonresorbable polymers including polyolefins, polyesters, polyimides, polyamides, polyacrylates, polyaryletherketones, fluropolymers, siloxane based polymers, and/or others. Polymers may include resorbable polymers including polyesters (e.g. lactide and glycolide), polyanhydrides, poly(aminoacid) polymers (e.g. tyrosine based polymers), and/or others. Other possible materials include nonresorbable and resorbable ceramics (e.g. hydroxyapatite and calcium sulfate) or biocompatible glasses. They may be made of homogenous materials or reinforced materials. If they are separate components joined together at the frangible junction, they may be made of different materials.

The suture may be any flexible member, natural or synthetic. For example, the suture may include wires, tapes, tissue derived strands, and other suitable members. The suture may be a monofilament or multi-filament structure. The suture may be absorbable or non-absorbable. Examples of absorbable suture materials include catgut, polyglycolic acid, polyglactin, poliglecaprone, polydioxanone, poly-4-hydroxybutyrate, and other absorbable suture materials. Examples of non-absorbable suture materials include silk, polypropylene, polyester, nylon, ultra-high molecular weight polyethylene (UHMWPE), and other non-absorbable suture materials. Preferably an UHMWPE suture is used for applications involving bone-to-bone and tissue-to-bone attachments due to its longevity and strength during the tissue-to-tissue healing process. If necessary, the suture may be tipped to aid in assembly of the flexible staple 100. The suture may be tipped using any appropriate tipping process including using, for example, heat, pressure, stretching, potting compounds, tape, shrink tubing, and other processes.

For example, an integral leg 106 and extension 120 may be machined from stainless steel with a round high strength braided suture secured to the legs by crimping. In another example, an integral leg 106 and extension 120 may be machined or molded from a polyaryletherketone, e.g. PEEK or PEEK carbon fiber composite, with a high strength suture tape secured to the legs by an adhesive. In another example, an integral leg 106 and extension 120 may be machined or molded from a reinforce polylactide polymer, e.g. glass fiber reinforced PLLA, and a resorbable suture secured to the legs 106 by a melt process. In another example, a leg 106 may be formed of a resorbable polymer and an extension 120 may be formed of a non-absorbable polymer with the leg and extension joined by press-fitting the components together and a high strength resorbable suture secured to the legs by a heat staking operation. The preceding examples serve merely to show some possible combinations and are not to be considered comprehensive. Other combinations are within the scope of the invention.

In the illustrative example of FIGS. 1-10, the legs 106 and extensions 120 are integrally formed by machining from implant grade stainless steel. The legs 106 have a barbed outer surface 136 with the barbs tapering inwardly distally so that the insertion force to drive the legs 106 into tissue is lower than the extraction force to remove the legs 106 from the tissue. The leg width is 2 mm and the leg length is 8 mm to 9 mm. The suture is a high strength #2 polyethylene multi-filament suture and the spanning length is 6 mm to 9 mm. The suture 130 is tipped to prevent the ends from unraveling by saturating the tips with a low viscosity cyanoacrylate and allowing it to cure so that the tips are stiff. A suture tip is threaded through the axial bore 140 of each leg 106 from the proximal end 110 to the distal end 112. To facilitate insertion of the suture, a relief groove 152 (FIGS. 6 and 7) is provided in each extension 120 to allow the suture tip to be inserted with a less acute bend. The leading end portion 138 of the leg is then crimped to secure the suture. Optionally the suture end may be extended out the distal end 112 of each leg 106 and the suture tip cut off so that the tipped portion is not a permanent part of the implant. Optionally, an adhesive may be placed on a portion of the suture prior to advancing the suture into the leg 106 so that the adhesive helps to bond the suture to the leg and the suture forms a permanent part of the assembly. For example, an adhesive may be applied to a portion of the suture near the proximal end before that portion of the suture is advanced into the leg. Alternatively, an adhesive may be applied to a portion of the suture that has been extended through the leg and that projects beyond the distal end 112 of the leg and then that portion retracted back into the leg. The suture is contained within each leg and exits the proximal end 110 so that it only contacts the proximal end and bridges between the legs at the proximal end. FIGS. 9 and 10 illustrate the staple 100 after the extensions 120 have been broken off.

Instruments may be provided to aid in performing a surgical procedure with the inventive flexible staple. The instruments may be reusable or disposable. The instruments may be provided with fixed feature spacing corresponding to the size of the staple or they may be adjustable such that a single instrument can accommodate multiple sizes. Preferably, at least some of the instruments are disposable and are packaged with the staple 100 to provide containment, protection, positioning, and ease of handling when the staple 100 is packaged, shipped, and used. The flexible staple 100 may be pressed into tissue by imparting a distally directed force on the extensions 120 and legs 106. For example, the extensions 120 and or legs 106 may be gripped, e.g. in a collet, and advanced into tissue. In another example, a force may be applied to the proximal end 122 of each extension. In the case of driving the legs 106 into bone, it is preferable to impact the proximal end 122 of each extension, e.g. by impacting the proximal ends 122 with a mallet. It is preferable to insert the staple 100 using a driver that shields the frangible junction 128 from bending loads during insertion to prevent unintended breaking of the frangible junction 128. Preferably, the driver allows selecting between driving the legs 106 simultaneously and independently. Preferably the driver provides for selectively retaining the legs 106 in a predetermined orientation relative to one another during handling. Preferably the driver provides gripping surfaces to facilitate gripping and manipulation of the staple 100, especially in the smaller sizes that may be used in small bone surgery.

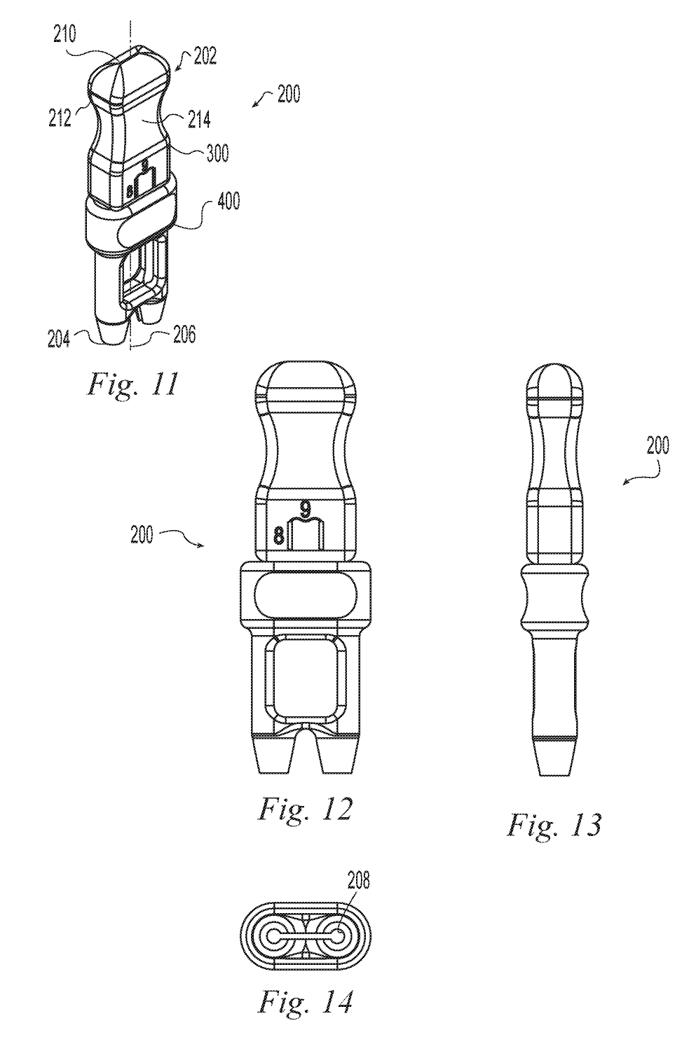

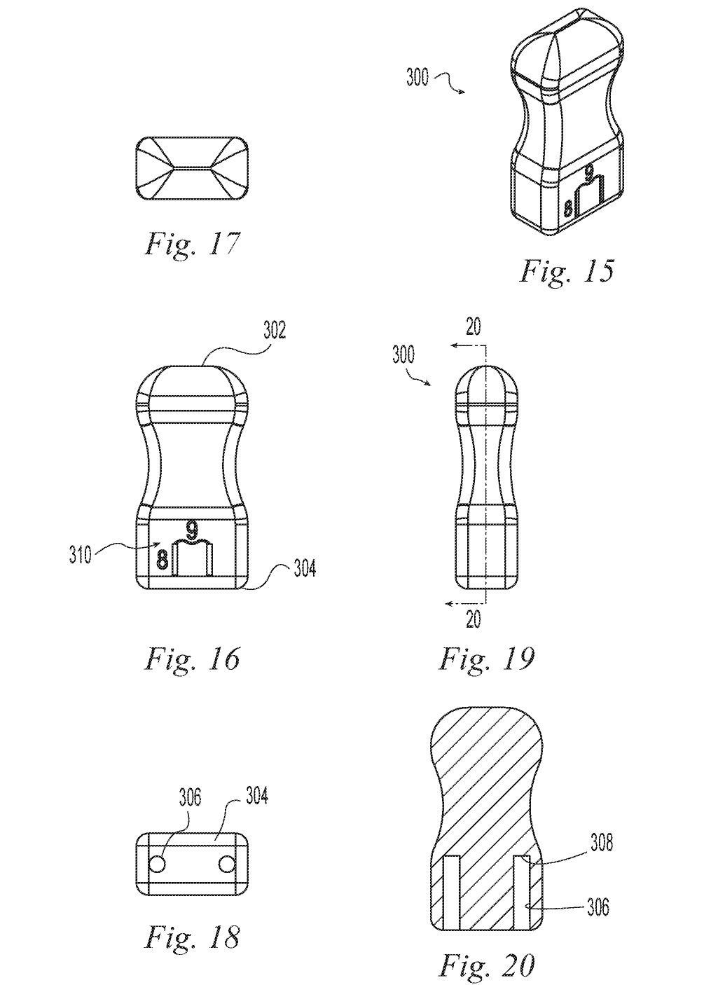

FIGS. 11-26 illustrate a driver 200 useable with the staple 100 of FIGS. 1-10. The driver 200 has a proximal end 202 and a distal end 204 spaced apart relative to a longitudinal axis 206. One or more axial passages 208 (FIG. 14) extend into the driver from the distal end toward the proximal end. Each passage 208 terminates within the driver 200 at a passage end wall. Each passage 208 receives an extension 120 in axial sliding relationship such that the proximal end of the extension abuts the passage end wall. The driver 200 includes an external striking surface 210 at the proximal end of the driver body. Impact force imparted to the external striking surface 210 is transmitted to the extension 120 and to the leg 106 to drive the leg into tissue. A grip 212 is formed near the proximal end 202 and includes a finger groove 214. Preferably the driver 200 extends over the frangible junction 128 to shield the frangible junction 128 from bending loads during insertion to prevent unintended breaking of the frangible junction 128. In the example of FIGS. 11-26, the driver 200 is a two-part driver including a driving body 300 and a sleeve body 400.

The driver body 300 serves to transmit insertion forces to the staple 100 and provide a grip for manipulating the staple. In the example of FIGS. 11-26, the driver body 300 has a proximal end 302 and a distal end 304. A pair of longitudinal passages 306 extends into the driver body 30 from the distal end 304 toward the proximal end. Each passage 306 terminates at a passage end wall 308 defining a driver face. Each passage 306 receives an extension 120 in axial sliding relationship with the proximal end of the extension abutting the driver face in axial, distal force transmitting relationship. The passages 306 are spaced apart a predetermined spacing corresponding to a desired spacing for the staple legs 106. Preferably, the spacing corresponds to the suture length between the legs 106 so that with the staple 100 engaged within the driver body 300, the suture is extended its full length. However, a driver body with narrower passage spacing may be provided if it is desired to insert the staple with some initial slack in the suture. In the example of FIGS. 11-26, the driver body 300 includes indicia 310 in the form of a schematic diagram of the staple legs 106 and suture 130 along with numeric indicators of the leg length and suture span between the legs in millimeters. These indicia provide positive identification of the particular staple to a user.

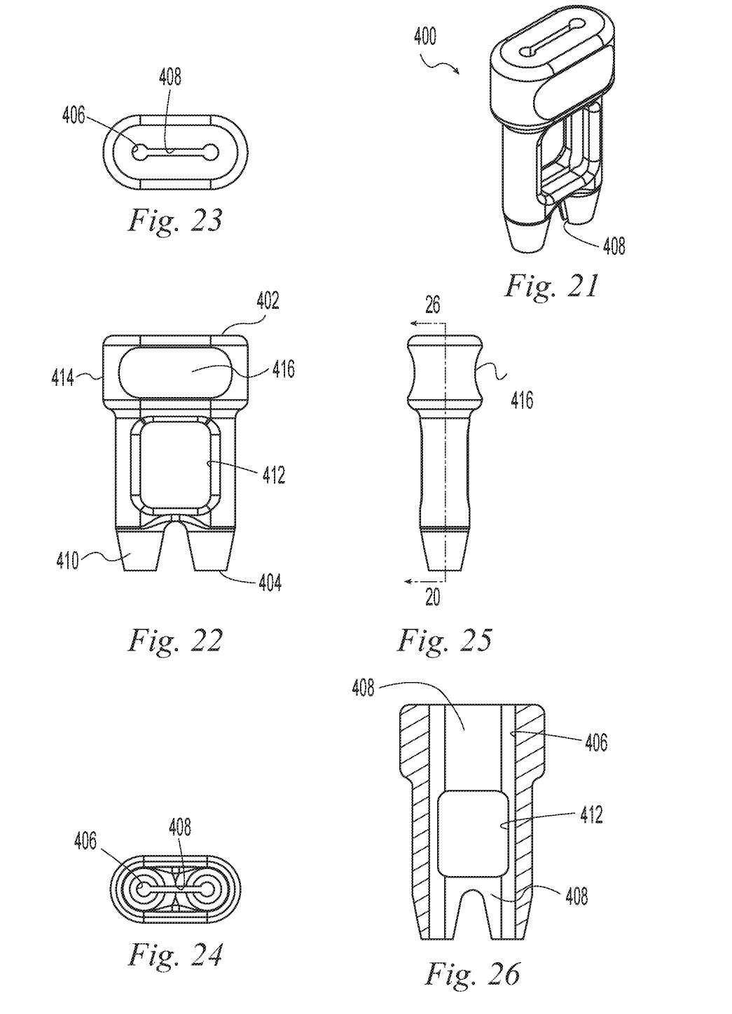

The sleeve body 400 serves to stabilize the legs 106 during insertion, protect the frangible junction 128, provide for manipulation of the staple, and together with the driver body 300, set the initial driving depth and provide containment and protection during shipping and storage. In the example of FIGS. 11-26, the sleeve body 400 has a proximal end 402 and a distal end 404. A pair of axial through passages 406 extends through the sleeve body 400 between the proximal and distal ends. Each passage 406 receives an extension 120 in axial sliding relationship with the proximal end 110 of the leg adjacent the distal end 404 of the sleeve body and the proximal end 122 of the extension 120 protruding from the proximal end 402 of the sleeve body 400. The passages 406 are spaced apart a predetermined spacing corresponding to a desired spacing for the staple legs 106 and corresponds to the passage spacing of the driver body 300. In the example of FIGS. 11-26, the sleeve body 400 includes a slot 408 connecting the through passages 406 at the distal end 404 for receiving the suture 130. Preferably, the slot 408 extends through the sleeve body 400 from the distal end 404 to the proximal end 402 as shown in FIGS. 21-26 so that the sleeve body 400 is freely positionable axially along the staple 100 or in other words the staple 100 may slide all the way through the sleeve body 400. With the slot 408 extending through the sleeve body 400, the sleeve body 400 and staple 100 may be assembled by inserting the staple 100 into the sleeve body 400 from either end. In the example of FIGS. 11-26, the distal end 404 of the sleeve body 400 includes first and second distally tapering extensions 410 coaxial with the passages 406 to provide for better illumination and viewing of the insertion site for the staple 100 and to provide clearance for tissue features such as highly sculpted bone at the insertion site. Likewise, in the example of FIGS. 11-26, a window 412 is formed through the sleeve body transverse to the passages 406. The window 412 also allows for better illumination and viewing of the surgical site. Preferably the sides of the sleeve body 400 are connected above and below the window for rigidity. In the example of FIGS. 11-26, the sleeve body 400 is generally in the form of two parallel tubes connected proximally and distally. The sleeve body 400 includes a grip 414 formed near the proximal end 402 including a finger groove 416.

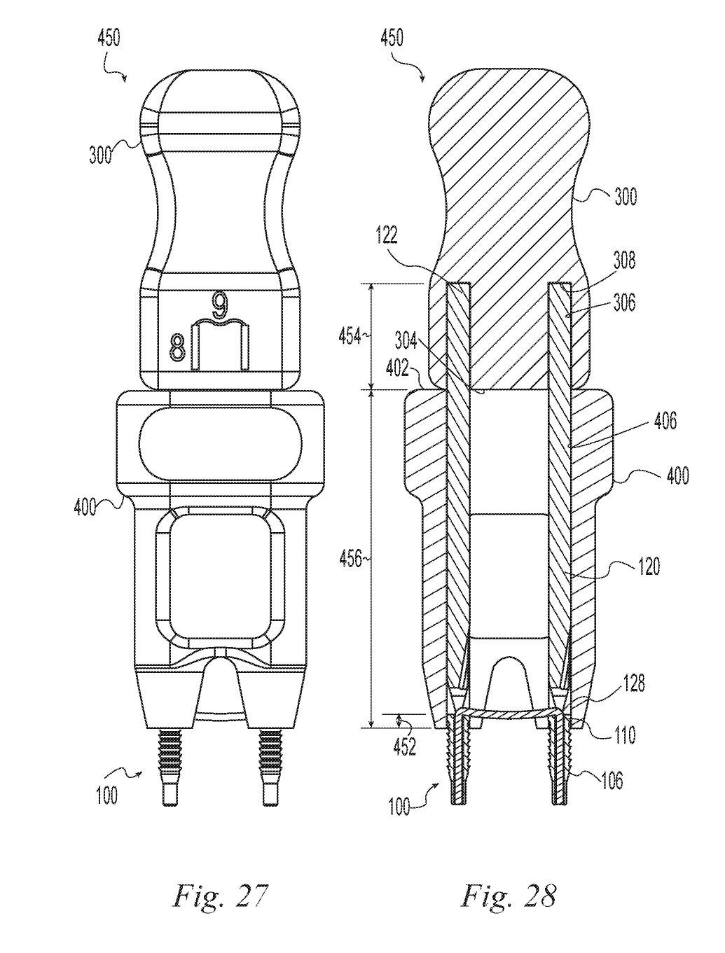

Referring to FIGS. 27 and 28, the driver body 300, sleeve body 400, and staple 100 are shown as an assembly 450. Preferably, this assembly 450 is how the staple and driver would be packaged for use. The assembly 450 is conveniently removed from its packaging ready for inserting the staple 100 into tissue. As shown in the example of FIGS. 11-28, the distal end 404 of the sleeve body 400 extends distally beyond the frangible junction 128 when the proximal end 122 of the extension 120 is contacting the end wall 308 of the passage 306 in the driver body 300 and the proximal end 402 of the sleeve body is contacting the distal end 304 of the driver body. Preferably, the distal end of the sleeve passage 406 engages the proximal end 110 of the leg 106 such that the sleeve body 300 bridges across the frangible junction 128 from the extension 120 to the leg 106 and shields the frangible junction 128 from bending forces. The length of engagement 452 of the passage 406 with the leg is determined by the depth 454 of the passage 306 in the driver body 300, the length 456 of the sleeve body 400, and the length 126 (FIG. 3) of the extension 120. The engagement length 452 determines how far the leg 106 will project from tissue when the assembly of FIGS. 27-28 is driven until the distal end 404 of the sleeve contacts the tissue. Preferably, the depth 454 of the passage 306 in the driver body 300, or in other words the projection of the extensions 120 from the sleeve body 400, is equal to or greater than the engagement length 452 so that the legs 106 can be driven at least flush with the surface of the tissue by removing the driver body 300 and driving the extensions further while they are supported by the sleeve body 400. More preferably the depth 454 is greater than the engagement length 452 so that the legs may be selectively driven below flush while the extensions are supported by the sleeve body 400.

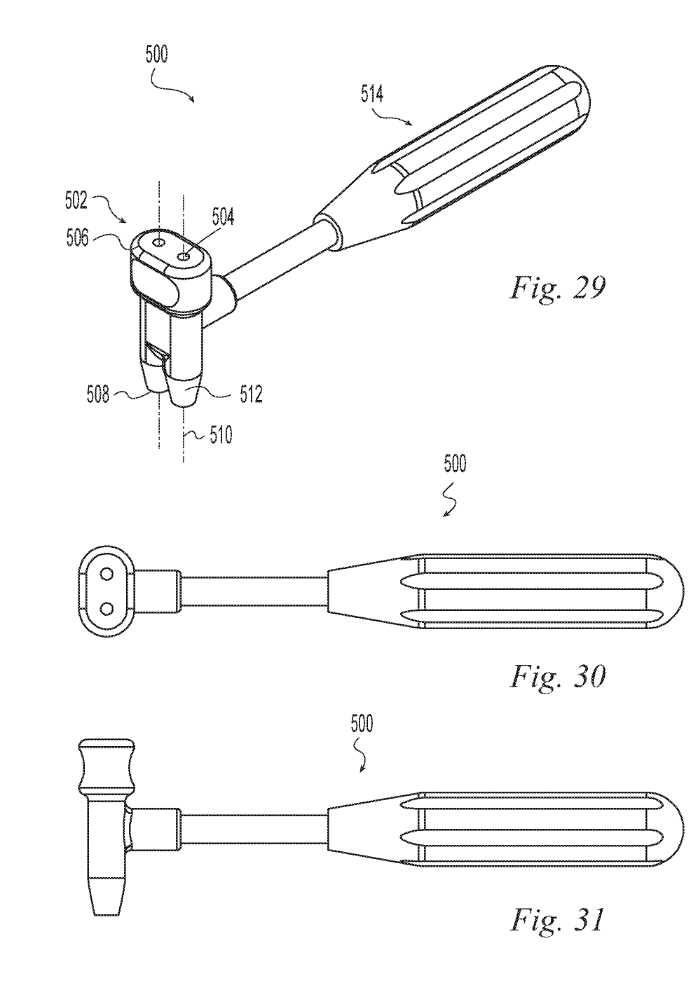

FIGS. 29-31 depict an illustrative example of a hole forming guide 500 for use with the staple and driver of FIGS. 1-28. The guide 500 functions to guide a hole forming instrument such as a drill bit, punch, wire, or other hole forming instrument to form holes in tissue that are spaced and aligned to receive the staple 100 in a desired position. The guide 500 includes a head 502 having passages 504 extending through the head from a proximal end 506 to a distal end 508 and defining passage axes 510. The passage axes 510 are spaced apart a distance corresponding to a desired spacing for inserting the staple legs 106 and preferably corresponding to the spacing of the driver. The axis spacing may be fixed or adjustable and the axes may be parallel or angled in one or more planes. In the illustrative example of FIGS. 29-31, the axes 510 are parallel and spaced apart a fixed distance equal to the length of a particular staple leg spacing. The distal end 508 of the guide head includes first and second distally tapering portions 512 coaxial with the passages 504 to provide for better illumination and viewing and to provide clearance for tissue features such as highly sculpted bone at the insertion site. The guide 500 includes a handle 514 extending from the head to aid a user in positioning and stabilizing the head 502 during use. The guide 500 may include indicia similar to that on the driver body 300 to indicate the distance between the axis 510.

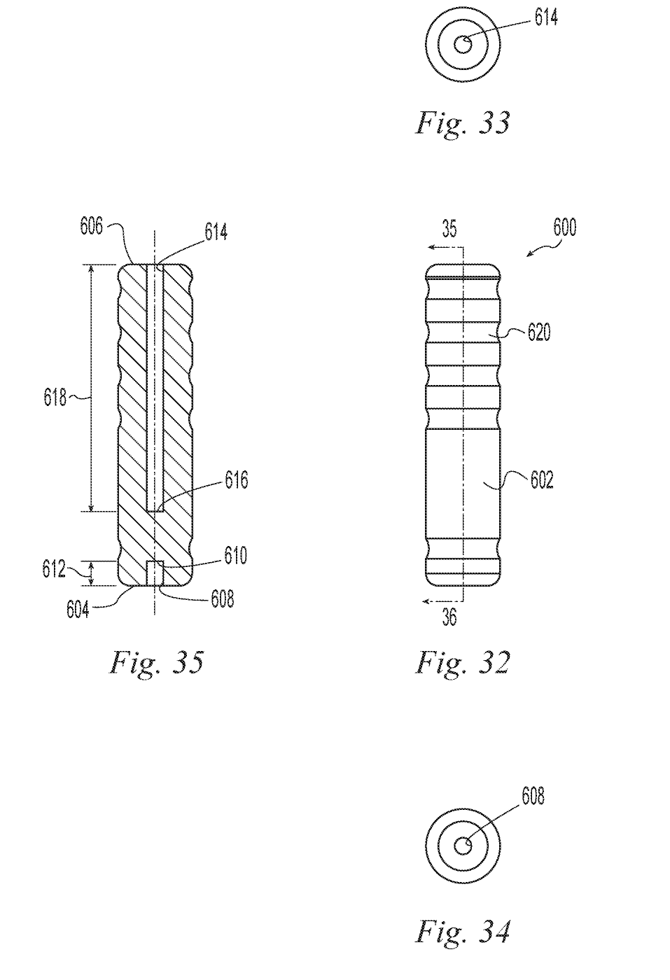

FIGS. 32-35 depict an illustrative example of a tamp 600 for use with the staple and driver of FIGS. 1-28. The tamp 600 functions to advance the staple legs further after they have been initially inserted using the driver 200. The tamp 600 allows independent driving of each staple leg 106 before the extension 120 has been broken off. The tamp 600 includes a tamp body 602 including a first end 604 and a second end 606. A first passage 608 extends into the tamp body 602 from the first end 604 toward the second end 606 and is sized to receive an extension 120. The first passage 608 terminates at a first passage end wall 610 defining a first tamp face for imparting a driving force to the proximal end of the extension 120. The first passage 608 has a length 612 from the first end to the first tamp face. The first passage 608 is operable to receive the proximal end of the extension 120 when the driver body is removed from the assembly of FIGS. 27-28. The length 612 of the first passage is sized so that with the extension 120 inserted into the first passage 608 with the extension contacting the first tamp face, the first end 604 of the tamp body is spaced proximally from the proximal end 402 of the sleeve body when the distal end 404 of the sleeve body is engaged with the proximal end 110 of a staple leg 106.

A second passage 614, extends into the tamp body 602 from the second end 606 toward the first end 604 and is size to receive an extension 120. The second passage 614 terminates at a second passage end wall 616 defining a second tamp face for imparting a driving force to the proximal end of the extension 120. The second passage has a length 618 from the second end 606 to the second passage end wall 616. The second passage 614 is operable to receive the proximal end of the extension 120 when the driver body and sleeve body are removed from the assembly of FIGS. 27-28. The length 618 of the second passage is sized so that with the extension inserted into the second tamp passage 614 with the extension contacting the second tamp face, the tamp body 602 covers more than one-half of the extension 120 and the second end 606 of the tamp body is spaced proximally from the frangible junction 128. The deep engagement of the second passage 614 with the extension 120 serves to reduce the bending moment that may be imparted to the staple 100 which reduces the likelihood of breaking the frangible junction 128 after the sleeve body 400 has been removed. The outer surface of the tamp preferably includes gripping features to enhance a user's grip on the tamp 600. In the illustrative example of FIGS. 32-35, grooves 620 are formed in the surface to enhance grip. Indicia may be provided to indicate which end contains the shallow passage and which end contains the deep passage. In the illustrative example of FIGS. 32-35, few grooves, in this example one, are positioned near the first end to indicate the shallow first passage 608 and many grooves, in this example 4, are positioned near the second end to indicate the deep second passage 614.

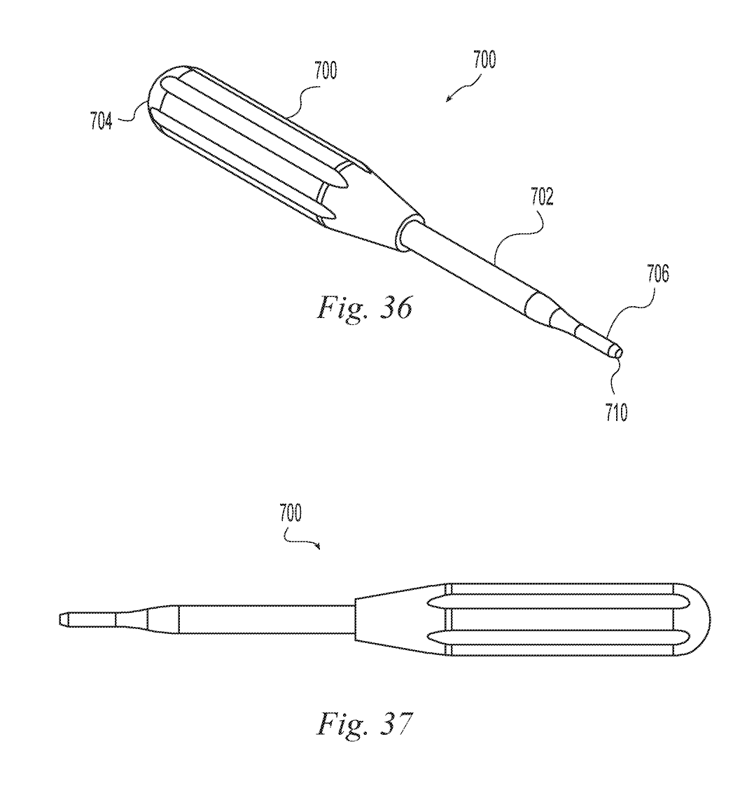

FIGS. 36 and 37 depict a staple leg setting tool 700 for independent driving of each staple leg 106 after the extension 120 has been broken off. The tool 700 includes a shaft 702 extending between a proximal end 704 and a distal end 706. A handle 708 is formed near the proximal end 704. A narrow tip 710 is formed at the distal end 706. The narrow tip preferably has a width equal to or smaller than the width of the proximal end of the staple leg 106. The narrow tip 710 is placed on the proximal end of the staple leg 106 and pushed or impacted to drive the staple leg further into tissue.

The flexible staple 100 and instruments of FIGS. 1-37 have applicability with a variety of tissues and procedures. For example, a first leg of the staple may be inserted into a first tissue portion and a second leg of the staple may be inserted into a second tissue portion with the suture extending between the first and second legs. After inserting the staple legs 106 the extensions 120 may be broken off leaving the staple legs and suture connected to the tissue. The legs 106 may be inserted simultaneously or independently. Preferably, the legs 106 are inserted simultaneously for a first distance and then independently to a desired final insertion depth. The suture may be tensioned by inserting at least one of the legs deep enough to pull the portion of the suture connected to the leg into the tissue.

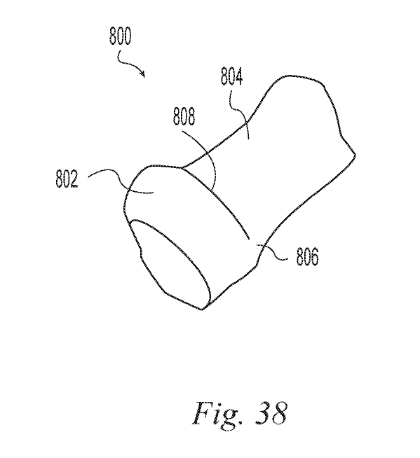

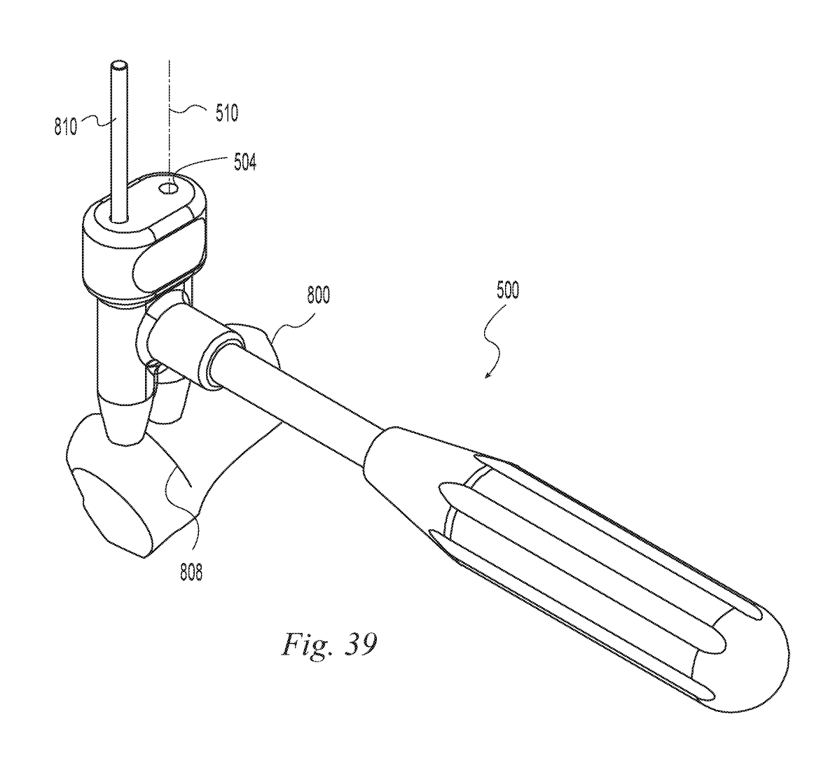

In order to demonstrate the use of the staple and instruments in more particular detail, an illustrative surgical procedure is depicted FIGS. 38-44. The illustrative procedure is applicable, for example, in securing an osteotomy. In particular, the illustrative procedure depicts a closing wedge osteotomy of a bone of a hand or foot. For example, the staple and instruments may be used to secure a medial closing wedge osteotomy of the proximal phalanx of the great toe.

FIG. 38 depicts a proximal phalanx bone 800 of a great toe. Converging saw cuts were made in the phalanx 800 to remove a wedge of bone between first and second bone portions 802, 804. The portions were pivoted relative to one another about a bone hinge 806 to close the gap created by removing the wedge of bone resulting in an osteotomy interface 808. The staple 100 will be used to secure the bone portions 802, 804 so that healing of the bone can occur at the interface 808.

FIG. 39 depicts the guide 500 positioned on the bone 800 with the guide passage axes 510 straddling the interface 808. A hole former 810, e.g. a K-wire, is guided in each passage 504 to form corresponding holes in the bone 800.

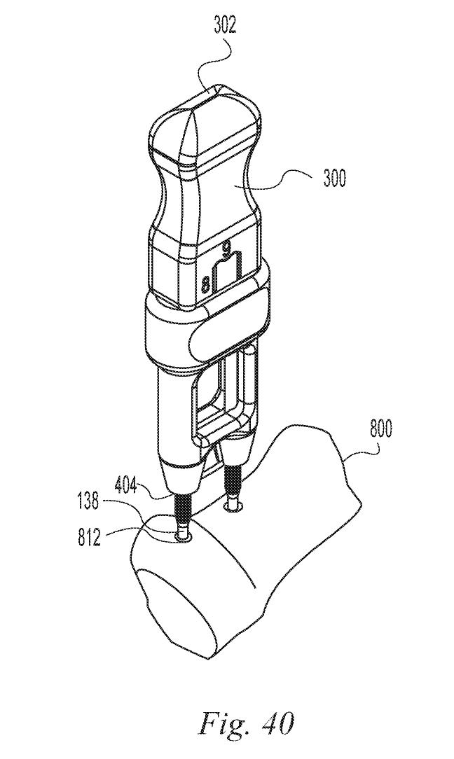

FIG. 40 depicts the staple and driver assembly 450 as the distal ends 138 of the legs 106 are started into the holes 812 formed in the bone. The staple legs are simultaneously advanced, e.g. by impacting the proximal end 302 of the driver body, until the distal end 404 of the sleeve body contacts the bone 800.

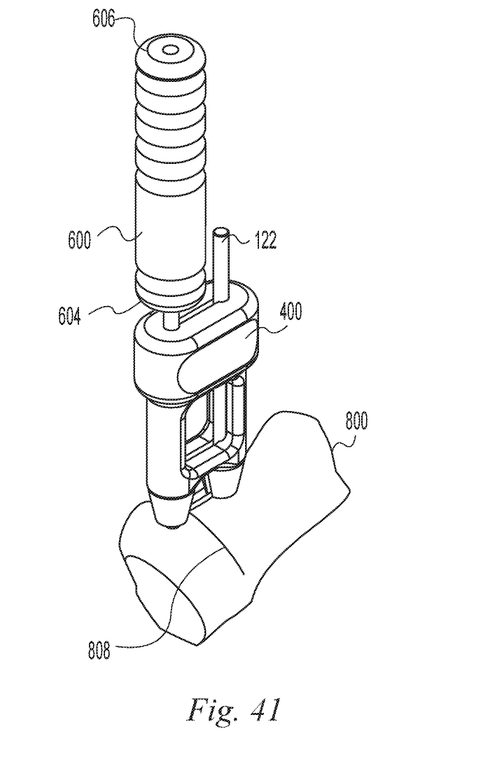

In FIG. 41, the driver body 300 has been removed leaving the proximal ends 122 of the extensions protruding from the sleeve body 400. If desired, at this point in the procedure, the shallow hole of the first end 604 of the tamp 600 is engaged with the proximal end 122 of one of the extensions and advanced, e.g. by impacting the second end 606 to independently advance one of the legs. The other leg may be similarly independently advanced. For example, the extension 120 and leg 106 may be advanced until the frangible junction 128 is at or below the bone surface or until the suture is tight and the osteotomy interface 808 is secured.

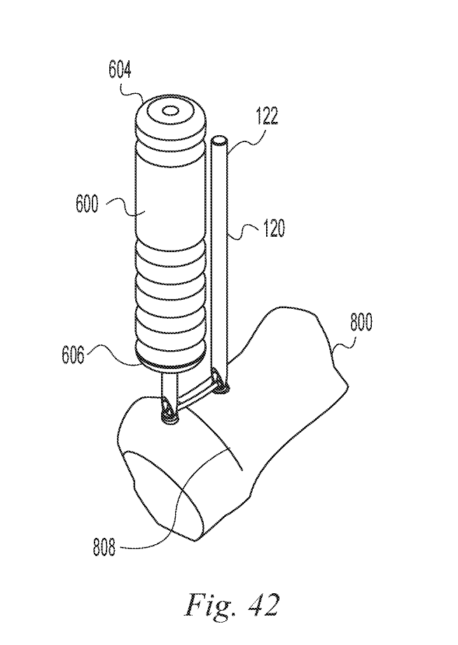

In FIG. 42, the sleeve body 400 has been removed leaving the extensions 120 protruding from the bone. If desired, at this point in the procedure, the deep hole of the second end 606 of the tamp 600 is engaged with the proximal end 122 of one of the extensions and advanced, e.g. by impacting the first end 604 to independently advance one of the legs. The other leg may be similarly independently advanced. For example, the extension 120 and leg 106 may be advanced until the frangible junction 128 is at or below the bone surface or until the suture is tight and the osteotomy interface 808 is secured. If further advancing of the legs 106 with the tamp 600 is not needed, instead of removing the sleeve body 400 entirely, it may be moved proximally up the extensions 120 so that it no longer covers the frangible junction 128 but is still engaged with the extensions 120 and the sleeve body 400 may be used as a handle to break the extensions away from the legs 106. Alternatively, the sleeve body may be removed and the extensions individually broken away.

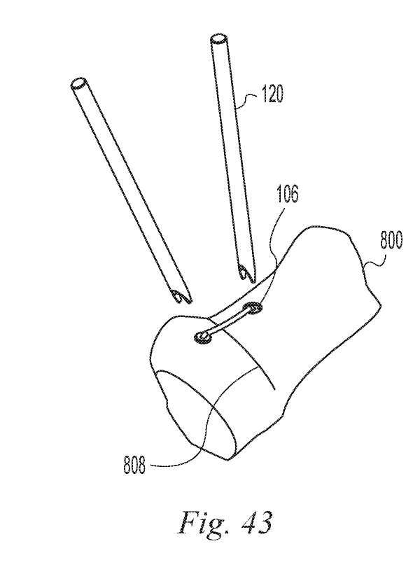

In FIG. 43, the extensions 120 have been broken away from the staple legs 106 by a bending motion.

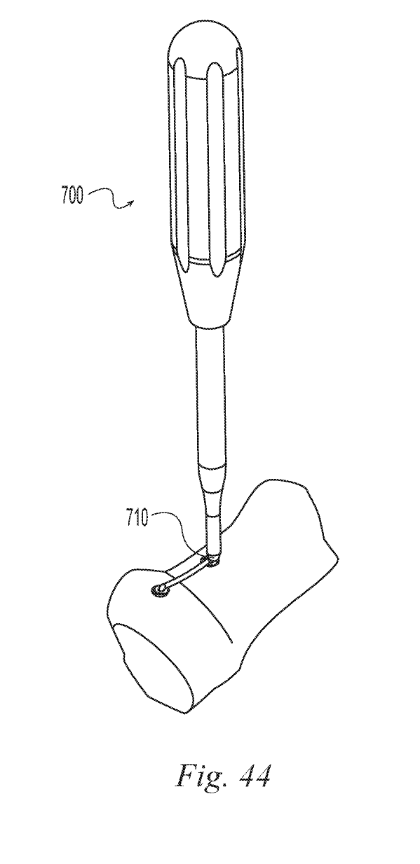

In FIG. 44, the tip 710 of the leg setting tool 700 is optionally engaged with the proximal end of a staple leg 106 to fine tune the insertion depth.

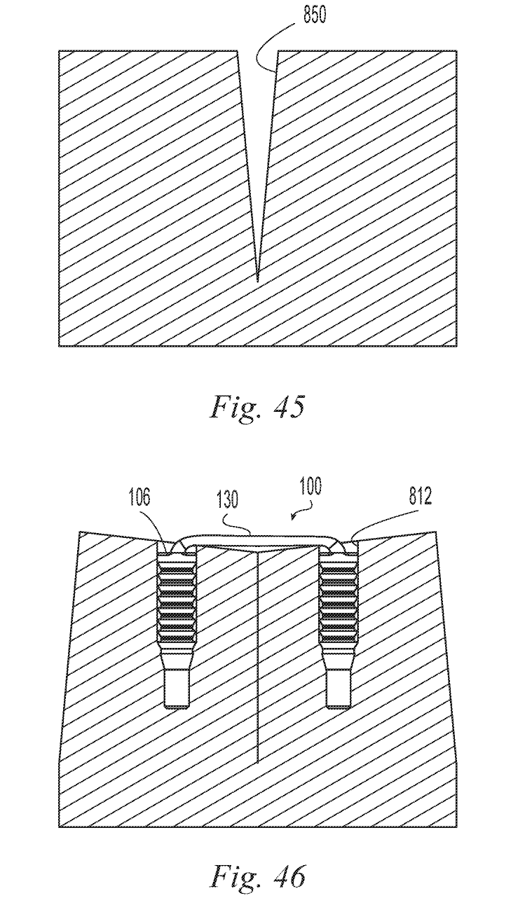

FIGS. 45 and 46 are cross sectional views of the staple used to secure a gap 850 in tissue. After the gap 850 is closed, the staple 100 is inserted as previously described to secure the tissue. As can be seen in FIG. 46, further advancing of the staple legs 106 will pull the suture 130 into the hole 812 behind the leg and thereby shorten the length of suture span between the holes and increase the tension in the suture and the tissue.

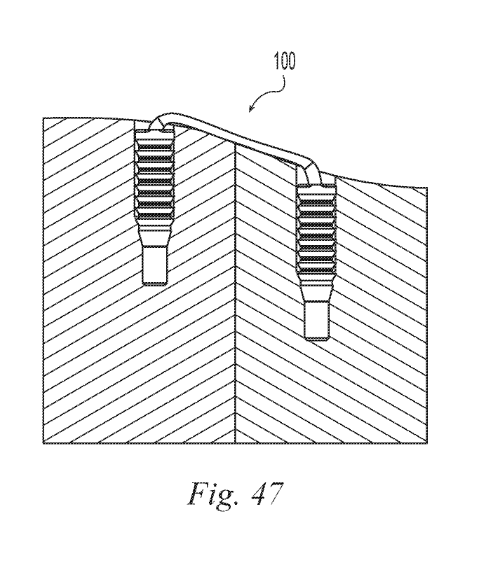

FIG. 47 illustrates the staple 100 accommodating irregular anatomy between the legs as the suture conforms to the irregular anatomy. The staple 100 with its headless legs and flexible suture bridge creates a low, non-irritating profile at the surgical site.

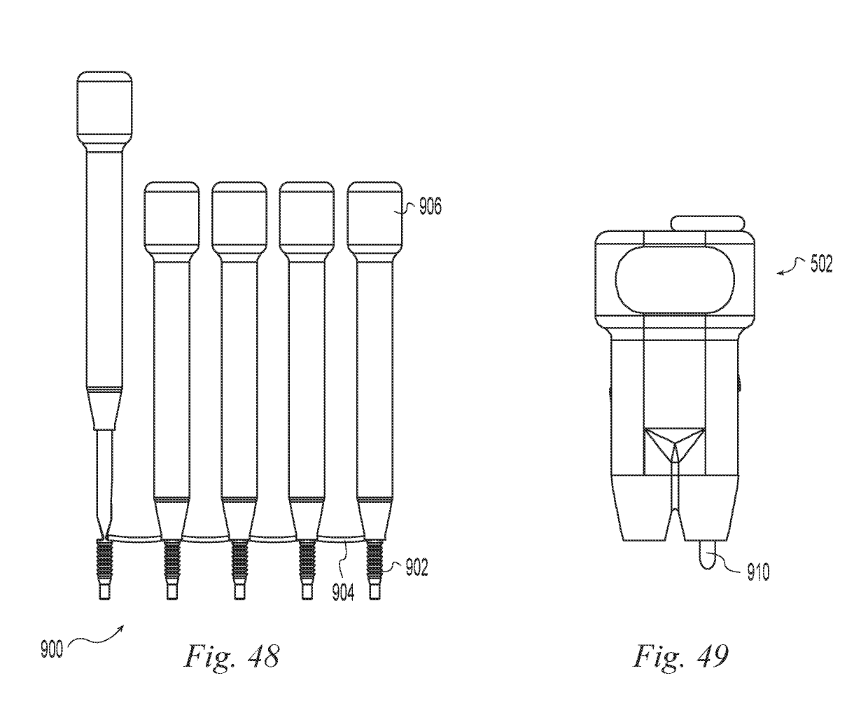

FIG. 48 illustrates a staple 900 with more than two legs 902 and a plurality of interconnecting sutures 904. Any number of legs and sutures may be connected. The chain of connected legs and sutures may be open as shown in FIG. 48 or the terminal legs may be joined to form a closed chain. Individual drivers 906 are shown engaged with each staple leg 902. However, the drivers may alternatively be joined together so that multiple legs may be simultaneously driven as shown in FIGS. 11-28. Individual drivers 906, as shown in FIG. 48 allow for arbitrary placement of the staple legs 902. The staple 900 may, for example, advantageously be used to secure soft tissue along an irregular bone margin.

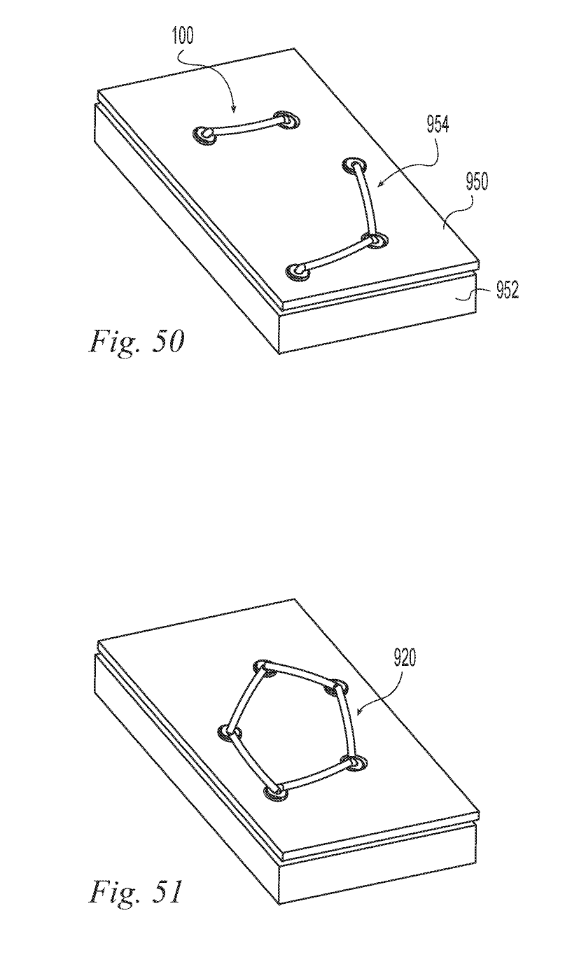

FIG. 49 illustrates the guide 500 with an indexing pin 910 extending from the distal end of the head 502. By engaging the indexing pin 910 in a previously formed hole, additional holes may be formed in the tissue by guiding a hole forming tool in the other passage in the guide head. The guide head 502 may be pivoted about the indexing pin 910 to guide the formation of additional holes at arbitrary angles but with a fixed spacing corresponding to the length of suture 904 extending between the legs 902. Alternatively, if the drivers 906 are joined in a fixed pattern, a guide may optionally be provided with a corresponding fixed hole pattern.

The staple 100 or 900 may also be used to attach a first member to tissue. For example, the first member may be soft tissue, a graft, an augmentation mesh, or other member to be attached to a tissue, e.g. bone. At least one leg 106, 902 is first passed through the first member and then into the tissue. For example, a first leg 106, 902 may be passed through soft tissue and then into a bone and then the second leg may be passed through the soft tissue and then into the bone. The second leg may also be inserted into bone without first passing through the soft tissue so that one leg passes through the soft tissue and the other one does not. In all of these examples, the suture 130, 904 extending between the legs passes over a portion of the first member. FIG. 50 illustrates the use of staples according to the invention in use to attach a first member 950 to a bone 952. For example, a staple 100 as shown in FIGS. 1-10 may be utilized by passing each of the two legs 106 through the first member 950 and into the bone as shown. In another example, a staple 954 with three legs joined by two suture portions is placed in an arbitrary pattern by forming holes in an arbitrary pattern using the guide head 502 and pin 910 as shown in FIG. 49. FIG. 51 illustrates the use of a five-legged staple 920 in which the suture portions connect the legs in a closed shape.

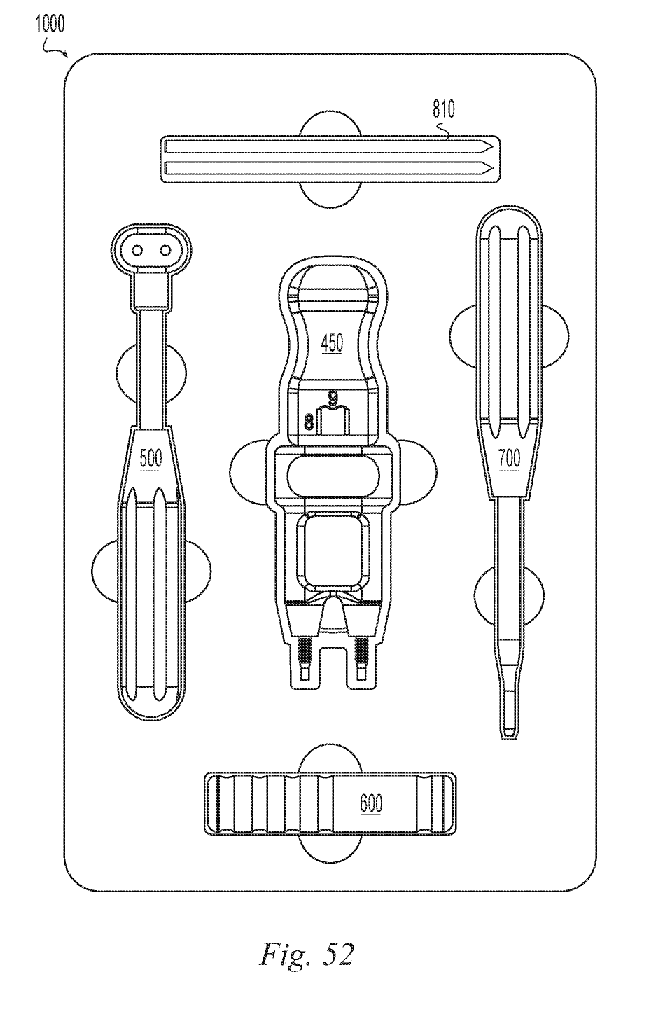

FIG. 52 illustrates a kit 1000 containing items useable in the described methods. The kit 1000 includes the assembly 450 including the staple 100 and driver 200, the guide 500, the tamp 600, the setting tool 700, and K-wires 810. The kit is preferably provided sterile packaged for single use. The instruments may, for example, be made of injection molded plastic.

Specific examples of the invention have been described. However, it will be apparent to one skilled in the art that various changes and substitutions may be made within the scope of the invention defined by the claims. Likewise, it is contemplated, and within the scope of the invention, that the various features of the illustrative examples may be interchanged among the illustrative examples.

The following are further examples of the invention.
1. A flexible bone staple, comprising:
   a first member having a proximal portion and a distal portion, the distal portion comprising a first leg having an elongate body extending along a first leg axis from a proximal end to a distal end and having a length measured between the proximal end and the distal end of the leg, the proximal portion comprising a first extension having an elongate body extending from a proximal end to a distal end and having a length measured between the proximal end and the distal end of the first extension, the distal end of the first extension being joined to the proximal end of the first leg at a first frangible junction in axial force transmitting relationship along the first leg axis; the first member being responsive to bending to break at the frangible junction and separate the first leg and first extension;
   a second member having a proximal portion and a distal portion, the distal portion comprising a second leg having an elongate body extending along a second leg axis from a proximal end to a distal end and having a length measured between the proximal end and the distal end of the leg, the proximal portion comprising a second extension having an elongate body extending from a proximal end to a distal end and having a length measured between the proximal end and the distal end of the second extension, the distal end of the second extension being joined to the proximal end of the second leg at a second frangible junction in axial force transmitting relationship along the second leg axis; the second member being responsive to bending to break at the frangible junction and separate the second leg and second extension; and
   a suture having a first portion fixed to the first leg and a second portion fixed to the second leg.
2. The flexible bone staple of example 1 wherein the first extension extends coaxial to the first leg axis and the second extension extends coaxial to the second leg axis.
3. The flexible bone staple of example 1 wherein the suture comprises a multifilament suture.
4. The flexible bone staple of example 1 wherein the first extension length is greater than the first leg length.
5. The flexible bone staple of example 4 wherein the first extension length is from two to five times the first leg length.
6. The flexible bone staple of example 1 wherein the suture contacts the first leg at the proximal end of the first leg and the suture contacts the second leg at the proximal end of the second leg.
7. The flexible bone staple of example 1 wherein the suture is fixed to the first and second legs by at least one of crimping, gluing, and tying.
8. The flexible bone staple of example 7 wherein the first leg has a first axial passage extending between the proximal end and the distal end of the first leg bounded by a sidewall and the second leg has a second axial passage extending between the proximal end and the distal end of the second leg bounded by a sidewall, a first portion of the suture being positioned within the first axial passage and a second portion of the suture being positioned within the second axial passage.
9. The flexible bone staple of example 8 wherein each of the first and second legs has a crimp formed in the sidewall to fix the suture within the axial passage.
10. The flexible bone staple of example 9 wherein each of the first and second legs further comprises an adhesive material in the axial passage.
11. The flexible bone staple of example 9 wherein the distal end of each of the first and second legs comprises a portion with a reduced outer diameter, relative to the proximal end of each leg, the suture extending from the proximal end to the portion with the reduced outer diameter in each of the first and second legs, a crimp being formed in the portion with the reduced outer diameter in each of the first and second legs.
12. The flexible bone staple of example 11 wherein each of the first and second legs comprises a barbed outer surface, the portion with the reduced outer diameter extending distally beyond the barbed outer surface.
13. The flexible bone staple of example 1 wherein each of the first and second members comprises a unitary structure in which the leg and extension are formed of one continuous material, the frangible junction comprising a connecting portion having a cross sectional area perpendicular to the leg axis, the leg having a cross sectional area perpendicular to the leg axis adjacent the connecting portion, the extension having a cross sectional area perpendicular to the leg axis adjacent the connecting portion, the cross sectional area of the connecting portion being less than the cross sectional area of the leg and less than the cross sectional area of the extension.
14. The flexible bone staple of example 1 further comprising a third member having a distal third leg and a proximal third extension, the third leg being joined to at least one of the first and second legs by a suture portion.
15. A method of making a flexible bone staple, comprising:
   utilizing a first member having a proximal portion and a distal portion, the distal portion comprising a first leg having an elongate body extending along a first leg axis from a proximal end to a distal end and having a length measured between the proximal end and the distal end of the leg, the first leg including a first axial passage extending from the proximal end of the first leg at least part-way to the distal end of the first leg, the proximal portion comprising a first extension having an elongate body extending from a proximal end to a distal end and having a length measured between the proximal end and the distal end of the first extension, the distal end of the first extension being joined to the proximal end of the first leg at a first frangible junction in axial force transmitting relationship along the first leg axis; the first member being responsive to bending to break at the frangible junction and separate the first leg and first extension;
   utilizing a second member having a proximal portion and a distal portion, the distal portion comprising a second leg having an elongate body extending along a second leg axis from a proximal end to a distal end and having a length measured between the proximal end and the distal end of the leg, the second leg including a second axial passage extending from the proximal end of the second leg at least part-way to the distal end of the second leg, the proximal portion comprising a second extension having an elongate body extending from a proximal end to a distal end and having a length measured between the proximal end and the distal end of the second extension, the distal end of the second extension being joined to the proximal end of the second leg at a second frangible junction in axial force transmitting relationship along the second leg axis; the second member being responsive to bending to break at the frangible junction and separate the second leg and second extension;
   passing a first portion of a suture into the axial passage of the first leg and fixing the first portion of the suture to the first leg; and
   passing a second portion of the suture into the axial passage of the second leg and fixing the second portion of the suture to the second leg.
16. The method of example 15 wherein fixing the first portion of the suture to the first leg comprises crimping the first leg.
17. The method of example 16 wherein fixing the first portion of the suture to the first leg further comprises introducing an adhesive into the first axial passage.
18. The method of example 16 wherein fixing the first portion of the suture to the first leg further comprises applying an adhesive to a portion of the suture and then moving the adhesive bearing portion of the suture into the first axial passage.
19. A flexible staple assembly including:
   a staple comprising:
      a first member having a proximal portion and a distal portion, the distal portion comprising a first leg having an elongate body extending along a first leg axis from a proximal end to a distal end and having a length measured between the proximal end and the distal end of the leg, the proximal portion comprising a first extension having an elongate body extending from a proximal end to a distal end and having a length measured between the proximal end and the distal end of the first extension, the distal end of the first extension being joined to the proximal end of the first leg at a first frangible junction in axial force transmitting relationship along the first leg axis; the first member being responsive to bending to break at the frangible junction and separate the first leg and first extension;
      a second member having a proximal portion and a distal portion, the distal portion comprising a second leg having an elongate body extending along a second leg axis from a proximal end to a distal end and having a length measured between the proximal end and the distal end of the leg, the proximal portion comprising a second extension having an elongate body extending from a proximal end to a distal end and having a length measured between the proximal end and the distal end of the second extension, the distal end of the second extension being joined to the proximal end of the second leg at a second frangible junction in axial force transmitting relationship along the second leg axis; the second member being responsive to bending to break at the frangible junction and separate the second leg and second extension; and
      a suture having a first portion fixed to the first leg and a second portion fixed to the second leg; and
   a driver receiving the proximal end of at least one of the first and second extensions in axial force transmitting relationship.
20. The flexible staple assembly of example 19 wherein the driver extends over the frangible junction.
21. The flexible staple assembly of example 19 wherein the driver comprises:
   a first component comprising:
      a sleeve body having a proximal end and a distal end, first and second longitudinal through passages extending through the sleeve body between the proximal and distal ends, the first and second through passages receiving the first and second extensions in axial sliding relationship with the proximal ends of the legs adjacent the distal end of the sleeve body and the proximal ends of the extensions protruding from the proximal end of the sleeve body;
   a second component comprising:
      a driver body having a proximal end and a distal end, first and second longitudinal passages extending into the driver body from the distal end toward the proximal end and each terminating at a passage end wall defining a driver face, the first and second passages receiving the first and second extensions in axial sliding relationship, the proximal ends of the first and second extensions abutting the driver faces in axial, distal force transmitting relationship.
22. The flexible staple assembly of example 21 wherein the sleeve body comprises a slot connecting the first and second through passages at the distal end, the slot receiving the suture.
23. The flexible staple assembly of example 22 wherein the slot extends through the sleeve body from the proximal end to the distal end, the staple being operable to slide all the way through the sleeve body.
24. The flexible staple assembly of example 21 wherein the sleeve body extends distally beyond the first and second frangible junctions when the extensions are contacting the driver faces of the first and second passages in the driver body and the proximal end of the sleeve body is contacting the distal end of the driver body.
25. The flexible staple assembly of example 24 wherein the distal end of the sleeve body comprises first and second distally tapering extensions, the first distally tapering extension engaging the proximal end of the first leg and the second distally tapering extension engaging the proximal end of the second leg.
26. The flexible staple assembly of example 21 wherein the driver body comprises an external striking surface at the proximal end of the driver body, the driver body being operable to transmit impact forces imparted to the external striking surface to the extensions in contact with the driver faces.
27. The flexible staple assembly of example 21 further comprising a tamp, the tamp comprising:
   a tamp body including a first end and a second end, a first passage extending into the tamp body from the first end toward the second end, the first passage terminating at a first passage end wall defining a first tamp face, the first passage having a length from the first end to the first tamp face, a second passage, extending into the tamp body from the second end toward the first end, the second passage terminating at a second passage end wall defining a second tamp face, the second passage having a length from the second end to the second tamp face, the second passage length being greater than the first passage length, the first passage being operable to receive the proximal end of the first extension when the driver body is removed from the assembly, the length of the tamp first passage being sized so that with the first extension inserted into first tamp passage until the first extension contacts the first tamp face the first end of the tamp body is spaced proximally from the proximal end of the sleeve body when the distal end of the sleeve body is engaged with the proximal end of the first leg; the second passage being operable to receive the proximal end of the first extension when the driver body and sleeve body are removed from the assembly, the length of the tamp second passage being sized so that with the first extension inserted into the second tamp passage until the first extension contacts the second tamp face the tamp covers more than one-half of the extension and the second end of the tamp body is spaced proximally from the frangible junction.
28. A method of using the flexible bone staple of example 1, the method comprising:
   inserting the first leg into a first bone portion;
   inserting the second leg into a second bone portion, the suture extending between the first and second legs;
   tensioning the suture by driving at least one of the first and second legs below the surface of the first or second bone portions; and
   breaking the first and second extensions away from the first and second legs.
29. The method of example 28 wherein the first and second legs are inserted simultaneously.
30. The method of example 28 wherein the first and second legs are first inserted simultaneously and then subsequently further inserted independently.
31. The method of example 28 wherein tensioning the suture moves the first and second bone portions together.
32. The method of example 28 wherein at least one of the first and second legs is first inserted through a member selected from the group consisting of soft tissue, graft, and mesh and then inserted into a bone portion.
33. A method of using the flexible bone staple assembly of example 21, the method comprising:

impacting the driver body to advance the first and second legs into first and second bone portions;
removing the driver body;
individually driving the first and second extensions to independently advance the first and second legs to independent insertion depths;
moving the sleeve body proximally relative to the legs; and
breaking the first and second extensions away from the first and second legs.
34. The method of example 33 wherein individually driving the first and second extensions comprises engaging at least one of the first and second extensions with a tamp having a first blind passage and impacting the tamp while the sleeve is engaged with the staple.
35. The method of example 34 further comprising removing the sleeve and engaging at least one of the first and second extensions with a second blind passage in the tamp and impacting the tamp.
36. A method of performing an osteotomy using the bone staple of example 1, the method comprising:
   cutting a bone to define first and second bone portions on either side of a cut;
   inserting the first leg into the first bone portion;
   inserting the second leg into the second bone portion, the suture extending between the first and second legs;
   securing the first and second bone portions together by driving at least one of the first and second legs below the surface of the first or second bone portions; and
   breaking the first and second extensions away from the first and second legs.
37. The method of example 36 wherein cutting a bone to define first and second bone portions comprises cutting the bone part-way through so that the first and second bone portions are joined by a third portion of bone.
38. The method example 37 wherein cutting a bone comprises cutting a medial wedge from a proximal phalanx of a great toe of a human foot.

What is claimed is:

1. A flexible bone staple, comprising:
a first member having a proximal portion and a distal portion, the distal portion comprising a first leg having an elongate body extending along a first leg axis from a proximal end to a distal end and having a length measured between the proximal end and the distal end of the leg, the proximal portion comprising a first extension having an elongate body extending from a proximal end to a distal end and having a length measured between the proximal end and the distal end of the first extension, the distal end of the first extension being joined to the proximal end of the first leg at a first frangible junction in axial force transmitting relationship along the first leg axis; the first member being responsive to bending to break at the frangible junction and separate the first leg and first extension;
a second member having a proximal portion and a distal portion, the distal portion comprising a second leg having an elongate body extending along a second leg axis from a proximal end to a distal end and having a length measured between the proximal end and the distal end of the leg, the proximal portion comprising a second extension having an elongate body extending from a proximal end to a distal end and having a length measured between the proximal end and the distal end of the second extension, the distal end of the second extension being joined to the proximal end of the second leg at a second frangible junction in axial force transmitting relationship along the second leg axis; the second member being responsive to bending to break at the frangible junction and separate the second leg and second extension; and
a suture having a first portion fixed to the first leg and a second portion fixed to the second leg.

2. The flexible bone staple of claim 1 wherein the first extension extends coaxial to the first leg axis and the second extension extends coaxial to the second leg axis.

3. The flexible bone staple of claim 1 wherein the first extension length is greater than the first leg length.

4. The flexible bone staple of claim 3 wherein the first extension length is from two to five times the first leg length.

5. The flexible bone staple of claim 1 wherein the suture contacts the first leg at the proximal end of the first leg and the suture contacts the second leg at the proximal end of the second leg.

6. The flexible bone staple of claim 1 wherein the suture is fixed to the first and second legs by at least one of crimping, gluing, and tying.

7. The flexible bone staple of claim 6 wherein the first leg has a first axial passage extending between the proximal end and the distal end of the first leg bounded by a sidewall and the second leg has a second axial passage extending between the proximal end and the distal end of the second leg bounded by a sidewall, a first portion of the suture being positioned within the first axial passage and a second portion of the suture being positioned within the second axial passage.

8. The flexible bone staple of claim 7 wherein each of the first and second legs has a crimp formed in the sidewall to fix the suture within the axial passage.

9. The flexible bone staple of claim 8 wherein the distal end of each of the first and second legs comprises a portion with a reduced outer diameter, relative to the proximal end of each leg, the suture extending from the proximal end to the portion with the reduced outer diameter in each of the first and second legs, a crimp being formed in the portion with the reduced outer diameter in each of the first and second legs.

10. The flexible bone staple of claim 9 wherein each of the first and second legs comprises a barbed outer surface, the portion with the reduced outer diameter extending distally beyond the barbed outer surface.

11. The flexible bone staple of claim 1 wherein each of the first and second members comprises a unitary structure in which the leg and extension are formed of one continuous material, the frangible junction comprising a connecting portion having a cross sectional area perpendicular to the leg axis, the leg having a cross sectional area perpendicular to the leg axis adjacent the connecting portion, the extension having a cross sectional area perpendicular to the leg axis adjacent the connecting portion, the cross sectional area of the connecting portion being less than the cross sectional area of the leg and less than the cross sectional area of the extension.

12. The flexible bone staple of claim 1 further comprising a third member having a distal third leg and a proximal third extension, the third leg being joined to at least one of the first and second legs by a suture portion.

13. A flexible staple assembly including:
a staple comprising:
a first member having a proximal portion and a distal portion, the distal portion comprising a first leg having an elongate body extending along a first leg axis from a proximal end to a distal end and having a length measured between the proximal end and the distal end of the leg, the proximal portion comprising a first extension having an elongate body extending from a proximal end to a distal end and having a length measured between the proximal end and the distal end of the first extension, the distal end of the first extension being joined to the proximal end of the first leg at a first frangible junction in axial force transmitting relationship along the first leg axis; the first member being responsive to bending to break at the frangible junction and separate the first leg and first extension;
a second member having a proximal portion and a distal portion, the distal portion comprising a second leg having an elongate body extending along a second leg axis from a proximal end to a distal end and having a length measured between the proximal end and the distal end of the leg, the proximal portion comprising a second extension having an elongate body extending from a proximal end to a distal end and having a length measured between the proximal end and the distal end of the second extension, the distal end of the second extension being joined to the proximal end of the second leg at a second frangible junction in axial force transmitting relationship along the second leg axis; the second member being responsive to bending to break at the frangible junction and separate the second leg and second extension; and
a suture having a first portion fixed to the first leg and a second portion fixed to the second leg; and
a driver receiving the proximal end of at least one of the first and second extensions in axial force transmitting relationship.

14. The flexible staple assembly of claim 13 wherein the driver extends over the frangible junction.

15. The flexible staple assembly of claim 13 wherein the driver comprises:
a first component comprising:
a sleeve body having a proximal end and a distal end, first and second longitudinal through passages extending through the sleeve body between the proximal and distal ends, the first and second through passages receiving the first and second extensions in axial sliding relationship with the proximal ends of the legs adjacent the distal end of the sleeve body and the proximal ends of the extensions protruding from the proximal end of the sleeve body;
a second component comprising:
a driver body having a proximal end and a distal end, first and second longitudinal passages extending into the driver body from the distal end toward the proximal end and each terminating at a passage end wall defining a driver face, the first and second passages receiving the first and second extensions in axial sliding relationship, the proximal ends of the first and second extensions abutting the driver faces in axial, distal force transmitting relationship.

16. The flexible staple assembly of claim 15 wherein the sleeve body comprises a slot connecting the first and second through passages at the distal end, the slot receiving the suture.

17. The flexible staple assembly of claim 16 wherein the slot extends through the sleeve body from the proximal end to the distal end, the staple being operable to slide all the way through the sleeve body.

18. The flexible staple assembly of claim 15 wherein the sleeve body extends distally beyond the first and second frangible junctions when the extensions are contacting the driver faces of the first and second passages in the driver body and the proximal end of the sleeve body is contacting the distal end of the driver body.

19. The flexible staple assembly of claim 15 wherein the driver body comprises an external striking surface at the proximal end of the driver body, the driver body being operable to transmit impact forces imparted to the external striking surface to the extensions in contact with the driver faces.

20. The flexible staple assembly of claim 15 further comprising a tamp, the tamp comprising:
a tamp body including a first end and a second end, a first passage extending into the tamp body from the first end toward the second end, the first passage terminating at a first passage end wall defining a first tamp face, the first passage having a length from the first end to the first tamp face, a second passage, extending into the tamp body from the second end toward the first end, the second passage terminating at a second passage end wall defining a second tamp face, the second passage having a length from the second end to the second tamp face, the second passage length being greater than the first passage length, the first passage being operable to receive the proximal end of the first extension when the driver body is removed from the assembly, the length of the tamp first passage being sized so that with the first extension inserted into first tamp passage until the first extension contacts the first tamp face the first end of the tamp body is spaced proximally from the proximal end of the sleeve body when the distal end of the sleeve body is engaged with the proximal end of the first leg; the second passage being operable to receive the proximal end of the first extension when the driver body and sleeve body are removed from the assembly, the length of the tamp second passage being sized so that with the first extension inserted into the second tamp passage until the first extension contacts the second tamp face the tamp covers more than one-half of the extension and the second end of the tamp body is spaced proximally from the frangible junction.

21. A method of using the flexible bone staple of claim 1, the method comprising:
inserting the first leg into a first bone portion;
inserting the second leg into a second bone portion, the suture extending between the first and second legs;
tensioning the suture by driving at least one of the first and second legs below the surface of the first or second bone portions; and
breaking the first and second extensions away from the first and second legs.

22. The method of claim 21 wherein the first and second legs are inserted simultaneously.

23. The method of claim 21 wherein the first and second legs are first inserted simultaneously and then subsequently further inserted independently.

24. The method of claim 21 wherein tensioning the suture moves the first and second bone portions together.

25. A method of using the flexible bone staple assembly of claim 21, the method comprising:
impacting the driver body to advance the first and second legs into first and second bone portions;
removing the driver body;
individually driving the first and second extensions to independently advance the first and second legs to independent insertion depths;
moving the sleeve body proximally relative to the legs; and
breaking the first and second extensions away from the first and second legs.

26. The method of claim 25 wherein individually driving the first and second extensions comprises engaging at least one of the first and second extensions with a tamp having a first blind passage and impacting the tamp while the sleeve is engaged with the staple.

27. The method of claim 26 further comprising removing the sleeve and engaging at least one of the first and second extensions with a second blind passage in the tamp and impacting the tamp.

28. A method of performing an osteotomy using the bone staple of claim 1, the method comprising:
cutting a bone to define first and second bone portions on either side of a cut;
inserting the first leg into the first bone portion;
inserting the second leg into the second bone portion, the suture extending between the first and second legs;
securing the first and second bone portions together by driving at least one of the first and second legs below the surface of the first or second bone portions; and
breaking the first and second extensions away from the first and second legs.

29. The method of claim 28 wherein cutting a bone to define first and second bone portions comprises cutting the bone part-way through so that the first and second bone portions are joined by a third portion of bone.

30. The method of claim 29 wherein cutting a bone comprises cutting a medial wedge from a proximal phalanx of a great toe of a human foot.
